(19) United States
(12) Patent Application Publication
(10) Pub. No.: US 2013/0231461 A1
(43) Pub. Date: Sep. 5, 2013

(54) METHOD FOR THE ISOLATION OF HIGH DENSITY LIPOPROTEIN

(71) Applicant: VASCULARSTRATEGIES LLC, Plymouth Meeting, PA (US)

(72) Inventors: Steven Adelman, Doylestown, PA (US); Heidi Collins, Hatboro, PA (US); Anthony Sulpizio, Secane, PA (US)

(73) Assignee: VascularStrategies LLC, Plymouth Meeting, PA (US)

(21) Appl. No.: 13/783,723

(22) Filed: Mar. 4, 2013

Related U.S. Application Data
(60) Provisional application No. 61/605,876, filed on Mar. 2, 2012.

(51) Int. Cl. C07K 14/775

(52) U.S. Cl. CPC C07K 14/775
USPC 530/359

(57) ABSTRACT

The present invention relates to an improved method for the isolation of high density lipoprotein (HDL) or HDL-like particles. The method of the present invention provides for the isolation of a more highly purified HDL that requires substantially less time than standard methods. The present invention relates to an improved method for the isolation of high density lipoprotein or HDL-like particles comprising the steps of precipitation of apoprotein B-containing proteins and a single ultracentrifugation to recover high density lipoprotein or HDL-like particles.

### CROSS-REFERENCE TO RELATED APPLICATIONS

This application claims priority to U.S. Provisional Application No. 61/605,876 filed Mar. 2, 2012, which is hereby incorporated by reference.

### FIELD OF THE INVENTION

The present invention relates to an improved method for the isolation of high density lipoprotein (HDL) or high density lipoprotein-like particles (HDL-like particles). The method of the present invention provides for the isolation of a more highly purified HDL that requires substantially less time than standard methods.

### BACKGROUND OF THE INVENTION

Lipoproteins are characterized based on their density, and major classes present in fasting subjects' plasma samples include very low density lipoprotein (VLDL), low density lipoprotein (LDL) and high density lipoprotein (HDL). Studied over a number of years, these lipoproteins have very different compositions, and importantly, functions. Lipoproteins are particles comprised of mixtures of lipids and proteins, and their buoyancy at specific densities is dependent on this composition. Specifically, lower density lipoproteins (VLDL and LDL) have been shown to contain high levels of neutral lipids such as cholesterol esters and triglycerides. Levels of these lipoproteins correlate with increased prevalence of cardiovascular disease with LDL and VLDL apparently acting to deliver these lipids to the vascular wall promoting the atherosclerotic process. In contrast, HDL, although buoyant and containing some neutral lipid, is somewhat more dense and typically comprised of approximately 50% protein. Numerous studies have demonstrated that elevated HDL levels correlate with protection from cardiovascular disease, and recent data suggests that it functions to remove cholesterol from the vascular wall to lessen the atherosclerotic process.

The correlations described above for promotion (LDL, VLDL) and protection (HDL) from cardiovascular disease by specific lipoproteins have been under intense study to better understand mechanisms and to identify potential targets for intervention. Specifically, protection by HDL, despite years of research, remains poorly understood. Resolving the mechanisms for protection has been hampered by an inability to rapidly and more simply obtain sufficient quantities for further study of individual samples of sufficiently purified HDL from study subjects, both in preclinical and clinical studies. Importantly, it has recently been demonstrated that function of HDL rather than simply the level may be the determining factor for its beneficial effects, and poorly functioning HDL may not be protective at all. These findings show a clear need for an improved method for isolation of purified HDL or HDL-like particles to enable the study from individual subjects for further study of composition and function.

Serum lipoprotein complexes have well defined densities and this characteristic can be exploited to separate and isolate the complexes from human serum. The densities of VLDL and LDL range from 0.95 to 1.063 g/ml while the densities of HDL and HDL-like particles range from 1.063 to 1.21 g/ml. In standard gradient density ultracentrifuge isolation protocols the lipoprotein classes are separated through the use of salt solutions of specifically defined densities and ultracentrifugation at specifically defined speeds to sequentially float and isolate the lipoproteins classes based on their buoyancy. At the conclusion of the spin the lipoproteins will be concentrated in layers (gradients) of the salt solution according to their density with the lowest density, most buoyant, complexes located in the upper gradient and the more dense lipoproteins concentrated in the bottom gradient.

Serum lipoproteins comprise a heterogeneous population of lipid-protein complexes that can be grouped into broad classes, very low (VLDL), low (LDL) and high (HDL) density, based on differences in particle density related to lipid and protein content. VLDL and LDL are composed of predominately lipid, while high density lipoproteins have a higher content of protein (˜50%). The density of LDL is between 1.006-1.063 g/ml while that of HDL and HDL-like particles is 1.063-1.21 g/ml. Classical methods to separate HDL from VLDL and LDL employ sequential density ultracentrifugation using potassium bromide salt solutions prepared with densities in the range of each lipoprotein class. One drawback of these methods for the preparation of purified HDL is that they require a minimum of two prolonged ultracentrifugation steps. The first step, which isolates VLDL and LDL from HDL, requires an 18 hour ultracentrifugation spin in d=1.063 g/ml KBr salt solution. The buoyant VLDL and LDL are concentrated in the upper layers of the salt gradient and can be easily removed leaving the less buoyant HDL along with other heavier proteins concentrated in the bottom layers. The HDL is then separated from other lipid-free serum proteins by performing a second ultracentrifugation step for 21 hours in d=1.21 g/ml KBr salt solution. The HDL is buoyant in this density salt solution thus at the end of the centrifugation, the upper layers of the gradient contains primarily HDL leaving other plasma proteins in the bottom fraction. This “sequential density gradient ultracentrifugation” procedure is the “gold standard” for isolation of HDL; however the prolonged time required for both ultracentrifugation steps and the need for multiple density adjustments clearly limits the throughput of the procedure.

In an attempt to reduce the intensive time and labor resources of the standard HDL isolation protocol, the present inventors have developed an improved method for the isolation and purification of HDL or HDL-like particles.

Current methods for isolation are either cumbersome or fail to sufficiently purify from other (non-lipoprotein) contaminating plasma proteins. Quantitative isolation of HDL has typically been done by two methods. The first labor intensive method involves two ultracentrifugation steps. The first step involves adjustment of plasma to a density of 1.063 g/ml and ultracentrifugation for 18 hours for removal of buoyant VLDL and LDL fractions. The second step requires readjustment of the density of the remaining plasma to a density of 1.21 g/ml followed by a second ultracentrifugation step. HDL which is buoyant at this density is then isolated from the remaining non-lipoprotein associated plasma proteins which do not float.

The second method involves the removal of apoprotein B-containing lipoproteins from a plasma or serum sample. Apoprotein B is the major protein present on LDL and VLDL, and is not found on HDL or HDL-like particles. Reagents such as polyethylene glycol have been shown to interact with apoprotein B, forming a complex that then will precipitate. With short-term, low speed centrifugation, this procedure allows for the removal of LDL and VLDL particles and enrichment of HDL or HDL-like particles. However, all other plasma proteins remain.

### SUMMARY OF THE INVENTION

The present invention relates to an improved method for isolating high density lipoprotein (HDL) or high density lipoprotein-like (HDL-like) particles from bodily fluids, such as plasma, serum, lymph, cerebral spinal fluid, or peritoneal lavage fluid or homogenized tissues that reduces the time of isolation of the HDL or HDL-like particles and increases the purity of the HDL.

In one embodiment, the present invention reduces the time of the initial step of removing the LDL and the VLDL. In another embodiment, the present invention relates to a method for isolation of high density lipoprotein or high density lipoprotein-like particles comprising adding one or more reagents to the plasma or serum, ultracentrifugation of the plasma or serum, and recovering the high density lipoprotein.

### DETAILED DESCRIPTION OF THE INVENTION

As used herein, the term “about” will be understood by persons of ordinary skill in the art and will vary to some extent on the context in which it is used. If there are uses of the term which are not clear to persons of ordinary skill in the art given the context in which it is used, “about” shall mean up to plus or minus 10% of the particular value.

As used herein, the term “high density lipoprotein (HDL)-like particles” will be understood by persons of ordinary skill in the art and shall mean particles with a lipid core and a density <1.21.

In an attempt to reduce the intensive time and labor resources of the standard HDL isolation protocol, the present inventors have investigated an alternative method for removing VLDL and LDL from bodily fluids or homogenized tissues that requires less than one hour and replaces the initial 18 hour ultracentrifugation step of the sequential density gradient ultracentrifugation method. The method of the present invention is based on the well-documented ability of reagents, such as polyethylene glycol (PEG), to selectively and irreversibly precipitate apoprotein B-containing lipoproteins (VLDL and LDL). The resultant insoluble complex is then easily removed by a 30 minute centrifugation step using a standard bench top microcentrifuge. The remaining supernatant contains HDL or HDL-like particles along with other lipid-free serum proteins and is comparable to the material obtained following the first 18 hour ultracentrifugation step in the sequential density gradient ultracentrifugation method. The HDL or HDL-like particles can then be selectively isolated by performing a 21 hour ultracentrifugation in a salt solution at a density of 1.21 as also performed in the sequential density gradient ultracentrifugation method.

The use of a reagent such as PEG to remove VLDL and LDL from bodily fluids or homogenized tissues results in a time and resource advantage over currently employed methods of HDL isolation. In order to confirm that PEG precipitation step is a valid alternative or replacement for the standard 18 hour ultracentrifugation in a salt solution with a density of 1.063 g/ml, a side by side comparison of the two methods of HDL isolation from human serum was performed and the yield and purity of HDL obtained from each method were compared. In addition, the use of PEG provides an advantage over the use of other LDL- and VLDL-precipitating reagents such as sulfated polysaccharides (such as dextran sulfate, sodium phosphotungstate, heparin) and divalent cations (such as manganese chloride, magnesium chloride), which require multiple centrifugation steps and a final dialysis step after ultracentrifugation to remove the trace amounts of the reagents.

By developing a system in which a reagent, such as polyethylene glycol, is used to remove the apoprotein B-containing lipoproteins (LDL, VLDL) combined with a single ultracentrifuge step to isolate the HDL or HDL-like particles from remaining plasma proteins, the present inventors have simplified the process and enhanced the overall purity of the recovered HDL or HDL-like particles. The present inventors have completed a series of studies demonstrating the present method simplifies the isolation of HDL and allows for the recovery of quantitative amounts of highly purified HDL or HDL-like particles.

In one embodiment, the present invention relates to a method for isolating high density lipoprotein or HDL-like particles from a bodily fluid or homogenized tissue comprising the steps of adding one or more reagents to the bodily fluid or homogenized tissue, ultracentrifugation of the bodily fluid or homogenized tissue, and recovering the high density lipoprotein or HDL-like particles.

In another embodiment, the present invention relates to a method for isolating high density lipoprotein or HDL-like particles from a bodily fluid or homogenized tissue comprising adding one or more reagents to the bodily fluid or homogenized tissue, adjusting the density of the bodily fluid or homogenized tissue, ultracentrifugation of the plasma or serum, and recovering the high density lipoprotein or HDL-like particles.

In a preferred embodiment, the present invention relates to a method for isolating high density lipoprotein or HDL-like particles from a bodily fluid or homogenized tissue comprising adding one or more reagents to the bodily fluid or homogenized tissue, ultracentrifugation of the bodily fluid or homogenized tissue, and recovering the high density lipoprotein or HDL-like particles, wherein the one or more reagents are not sulfated polysaccharides.

In another embodiment, the present invention relates to a method for isolating high density lipoprotein or HDL-like particles from bodily fluid or homogenized tissue comprising adding one or more reagents to the bodily fluid or homogenized tissue, ultracentrifugation of the bodily fluid or homogenized tissue, and recovering the high density lipoprotein or HDL-like particles, wherein the one or more reagents precipitate apoprotein B-containing proteins.

In another embodiment, the present invention relates to a method for isolating high density lipoprotein or HDL-like particles from bodily fluid or homogenized tissue comprising adding one or more reagents to the bodily fluid or homogenized tissue, ultracentrifugation of the bodily fluid or homogenized tissue, and recovering the high density lipoprotein or HDL-like particles, wherein the one or more reagents precipitate LDL and VLDL.

In one embodiment, the present invention relates to a method for isolating high density lipoprotein or HDL-like particles from bodily fluid or homogenized tissue comprising adding one or more reagents to the bodily fluid or homogenized tissue to precipitate apoprotein B-containing proteins from the bodily fluid or homogenized tissue, adjusting the density of the bodily fluid or homogenized tissue, ultracentrifugation of the bodily fluid or homogenized tissue and recovering the high density lipoprotein or HDL-like particles.

In one embodiment, the present invention relates to a method for isolating high density lipoprotein or HDL-like particles from bodily fluid or homogenized tissue comprising adding one or more reagents to the bodily fluid or homogenized tissue to precipitate apoprotein B-containing proteins from the bodily fluid or homogenized tissue, adjusting the density of the bodily fluid or homogenized tissue with potassium bromide, ultracentrifugation of the bodily fluid or homogenized tissue and recovering the high density lipoprotein or HDL-like particles.

In another embodiment, the present invention relates to a method for isolating high density lipoprotein or HDL-like particles from bodily fluid or homogenized tissue comprising adding one or more reagents to the bodily fluid or homogenized tissue to precipitate apoprotein B-containing proteins from the bodily fluid or homogenized tissue, adjusting the density of the plasma or serum to 1.21 g/ml, ultracentrifugation of the bodily fluid or homogenized tissue, and recovering the high density lipoprotein or HDL-like particles.

In another embodiment, the present invention relates to a method for isolating high density lipoprotein or HDL-like particles from bodily fluid or homogenized tissue comprising adding one or more reagents to the bodily fluid or homogenized tissue to precipitate apoprotein B-containing proteins from the bodily fluid or homogenized tissue, adjusting the density of the bodily fluid or homogenized tissue to 1.21 g/ml with potassium bromide, ultracentrifugation of the bodily fluid or homogenized tissue, and recovering the high density lipoprotein or HDL-like particles.

In one embodiment, the present invention relates to a method for isolating high density lipoprotein or HDL-like particles from a bodily fluid or homogenized tissue comprising adding one or more reagents to the bodily fluid or homogenized tissue, centrifuging the bodily fluid or homogenized tissue to remove apoprotein B-containing proteins, ultracentrifugation of the bodily fluid or homogenized tissue and recovering the high density lipoprotein or HDL-like particles.

In another embodiment, the present invention relates to a method for isolating high density lipoprotein or HDL-like particles from a bodily fluid or homogenized tissue comprising adding one or more reagents to the bodily fluid or homogenized tissue, centrifuging the bodily fluid or homogenized tissue to precipitate apoprotein B-containing proteins, adjusting the density of the bodily fluid or homogenized tissue, ultracentrifugation of the bodily fluid or homogenized tissue and recovering the high density lipoprotein or HDL-like particles.

In another embodiment, the present invention relates to a method for isolating high density lipoprotein or HDL-like particles from a bodily fluid or homogenized tissue comprising adding one or more reagents to the bodily fluid or homogenized tissue to precipitate apoprotein B-containing proteins, centrifuging the bodily fluid or homogenized tissue to remove the apoprotein B-containing proteins, ultracentrifugation of the bodily fluid or homogenized tissue, and recovering the high density lipoprotein or HDL-like particles.

In another embodiment, the present invention relates to a method for isolating high density lipoprotein or HDL-like particles from a bodily fluid or homogenized tissue comprising adding one or more reagents to the bodily fluid or homogenized tissue to precipitate apoprotein B-containing proteins, centrifuging the bodily fluid or homogenized tissue to remove the apoprotein B-containing proteins, adjusting the density of the bodily fluid or homogenized tissue, ultracentrifugation of the bodily fluid or homogenized tissue and recovering the high density lipoprotein or HDL-like particles.

In another embodiment, the present invention relates to a method of isolating high density lipoproteins or HDL-like particles from bodily fluid or homogenized tissue comprising adding one or more reagents to the bodily fluid or homogenized tissue to precipitate apoprotein B-containing proteins, centrifugation of the bodily fluid or homogenized tissue to pellet the apoprotein B-containing proteins, adjusting the density of the apoprotein B-containing proteins-free bodily fluid or homogenized tissue, ultracentrifugation of the apoprotein B-containing proteins-free bodily fluid or homogenized tissue, and recovering the high density lipoproteins or HDL-like particles.

In one embodiment of the present invention, the method comprises only one binding and precipitation step. In another embodiment of the present invention, the method comprises only one step of addition of one or more reagents.

In one embodiment of the method of the present invention, the high density lipoprotein or HDL-like particles is not precipitated from the bodily fluid or homogenized tissue after precipitation of the apoprotein B-containing proteins. In another embodiment of the method of the present invention, the high density lipoprotein or HDL-like particles is not precipitated from the bodily fluid or homogenized tissue after precipitation of the apoprotein B-containing proteins.

In an another embodiment, the present invention relates to a method for isolating high density lipoprotein or HDL-like particles from a bodily fluid or homogenized tissue comprising ultracentrifugation to remove non-lipoprotein associated components of the bodily fluid or homogenized tissue, and then adding one or more reagents to the bodily fluid or homogenized tissue to bind and precipitate the apo B-containing lipoproteins, and recovering the high density lipoproteins or high density lipoprotein-like particles.

In an alternate embodiment, the present invention relates to a method for isolating high density lipoprotein or HDL-like particles from a bodily fluid or homogenized tissue comprising ultracentrifugation at a density of 1.21 gm/ml to remove non-lipoprotein associated components of bodily fluid or homogenized tissue, and then adding one or more reagents to the bodily fluid or homogenized tissue to bind and precipitate the apo B-containing lipoproteins, and recovering the high density lipoproteins or high density lipoprotein-like particles.

In one embodiment of the present invention, high density lipoprotein or HDL-like particles can be isolated from bodily fluids including, but not limited to, plasma, serum, cerebral spinal fluid, lymph and peritoneal lavage fluid. In another embodiment of the present invention, high density lipoprotein or HDL-like particles can be isolated from homogenized tissues including, but not limited to, liver, kidney and vascular tissue/atherosclerotic plaques. In one embodiment of the present invention, the bodily fluids can be human. In another embodiment of the present invention, the bodily fluids can be non-human. In one embodiment of the present invention, the homogenized tissues can be human. In another embodiment of the present invention, the homogenized tissues can be non-human.

In one embodiment, the reagents used in the present invention are added to a bodily fluid or homogenized tissue to bind and precipitate apoprotein B-containing proteins. The reagents that may be used in the present invention include, but are not limited to, polyethylene glycol, polyanions, polyanions in combination with divalent cations, antibodies, tetracycline and combinations thereof. The polyanions that may be used in the present invention include, but are not limited to, dextran sulfate, manganese chloride, sodium phosphotungstate, magnesium chloride and combinations thereof. In some embodiments, the polyanions are not sulfated polysaccharides.

In another embodiment of the present invention, apoprotein B-containing proteins can be removed by antibodies to apoprotein B. In another embodiment, apoprotein B-containing proteins can be removed by cross-linked antibodies to apoprotein B.

In one embodiment, the density of the HDL-containing supernatant or HDL-like particle-containing supernatant can be adjusted with salts including, but not limited to, potassium bromide, sodium bromide and combinations thereof.

In one embodiment of the present invention, the ultracentrifugation step is a single ultracentrifugation. In one embodiment of the present invention, the ultracentrifugation is performed at 4° C. In one embodiment of the present invention, the ultracentrifugation is performed from about 16 hours to about 48 hours. In one embodiment of the present invention, the ultracentrifugation is performed for 16 hours. In one embodiment of the present invention, the ultracentrifugation is performed for 17 hours. In one embodiment of the present invention, the ultracentrifugation is performed for 18 hours. In another embodiment of the present invention, the ultracentrifugation is performed for 19 hours. In another embodiment of the present invention, the ultracentrifugation is performed for 20 hours. In a preferred embodiment of the present invention, the ultracentrifugation is performed for 21 hours. In one embodiment of the present invention, the ultracentrifugation is performed for 48 hours.

The method of the present invention can be performed on multiple samples simply depending on the size of the ultracentrifuge rotor, the number of tubes allowed and the timing dependent on the rotor utilized. The method of the present invention would allow for isolation of samples from multiple subjects, thus allowing for direct comparisons to be made with material obtained within a single isolation procedure.

### EXAMPLES

The invention now being generally described, it will be more readily understood by reference to the following examples which are included merely for purposes of illustration of certain aspects and embodiments of the present invention and are not intended to limit the invention.

Serum lipoprotein complexes have well defined densities and this characteristic can be exploited to separate and isolate the complexes from human serum. The densities of VLDL and LDL range from 0.95 to 1.063 g/ml while the densities of HDL or HDL-like particles range from 1.063 to 1.21 g/ml. In standard gradient density ultracentrifuge isolation protocols the lipoprotein classes are separated through the use of salt solutions of specifically defined densities and ultracentrifugation at specifically defined speeds to sequentially float and isolate the lipoproteins classes based on their buoyancy. At the conclusion of the spin the lipoproteins will be concentrated in layers (gradients) of the salt solution according to their density with the lowest density, most buoyant, complexes located in the upper gradient and the more dense lipoproteins concentrated in the bottom gradient. This method which, for the purposes of this document, is referred to a Protocol #1 is the gold standard for obtaining HDL from serum and requires an initial 18 hour ultracentrifugation to segregate HDL from VLDL and LDL followed by a second 21 hour long ultracentrifugation to purify the HDL by removing all other serum proteins present in the d>1.21 fraction.

The second method, referred to as Protocol #2, describes one embodiment of the present invention, which substantially shortens the HDL or HDL-like particle isolation procedure by eliminating the initial 18 hour ultracentrifugation step at d=1.063 g/ml gradient; replacing it with a 30 minute polyethylene glycol (PEG) precipitation to remove VLDL and LDL from serum. The next step of the method, ultracentrifugation at density of 1.21 is the same as described for Protocol #1.

Protocol 1: Gradient Density Isolation of HDL from Serum Using Sequential Ultracentrifugation Spins

Determining the Density of Starting Human Serum and Adjusting the Density to 1.063 with Potassium Bromide (KBr).

The density of a commercial source of pooled human serum (Bioreclamation, LLC) was determined by weighing three separate 1 ml aliquots of the serum and using the average of the 3 weights as the density. The density of human serum using this method was 1.0220 g/ml. A 1.5 ml aliquot of the material was used in this portion of the study. In preparation for the first gradient separation step, the density of the 1.5 ml of serum was adjusted to a density 1.063 with KBr. The amount of KBr needed to achieve this density was calculated using the following formula:

$$Grams of KBr = \frac{Initial Volume ( Final Density - Starting Density )}{1 - ( 0.298 ( Final Density ) )}$$

The density of 1.5 ml of human serum was increased from the starting density of 1.0220 to the final density of 1.063 by solubilizing 90 mg of KBr in the serum.

Separating VLDL and LDL from HDL by Ultracentrifugation in d=1.063 g/ml Salt Gradient.

The 1.5 ml of d=1.063 g/ml serum was pipetted into the bottom of a 10 ml polycarbonate ultracentrifuge tube. The serum was then overlayed with 8.5 ml of the d=1.063 g/ml KBr salt solution and the tube was placed into a type Ti70 rotor and centrifuged 41,800 rpm for 18 hours at 4° C. in a Beckman Model L80 Ultracentrifuge.

At the completion of the centrifugation, the top 5 ml of the KBr gradient containing lipoproteins with density <1.063 g/ml (i.e. VLDL and LDL) was removed in 1 ml layers and stored at 4° C. in Eppendorf tubes for future analysis if required. The remaining bottom 5 ml containing the HDL fraction was stirred and removed to a labeled 5 ml polypropylene tube.

Adjusting the Density of the Bottom 5 ml Fraction of the Gradient (Containing HDL) to d=1.21 g/ml with KBr.

The density of the bottom 5 ml fraction obtained from the first ultracentrifugation was determined by weighing five separate 0.2 ml aliquots and using the average of the 5 weights as the density. The density of the bottom fraction using this method was 1.041 g/ml. In preparation for the second gradient separation ultracentrifugation, the density of 4.5 ml (0.5 ml was reserved for other analysis) was adjusted to a density 1.21 g/ml with KBr. The amount of KBr needed to achieve this density was calculated using the following formula:

$$Grams of KBr = \frac{Initial Volume ( Final Density - Starting Density )}{1 - ( 0.318 ( Final Density ) )}$$

The density of the 4.5 ml of bottom fraction was increased from the starting density of 1.041 to the final density of 1.063 by solubilizing 1.23 mg of KBr in the 4.5 ml.

Purification of HDL by Ultracentrifugation in d=1.21 g/ml Salt Gradient.

The 4.5 ml of d=1.21 g/ml adjusted bottom fraction was transferred to the bottom of a 10 ml polycarbonate ultracentrifuge tube and was overlayed with 5.5 ml of d=1.21 g/ml salt gradient solution. The tube was placed in a type Ti70 rotor and centrifuged at 39,100 rpm for 21 hours at 4° C. in a Beckman Model L80 Ultracentrifuge. At the conclusion of the centrifugation, the 10 ml was progressively removed in 1 ml increments from top to bottom and the density of each gradient fraction was determined by weighing 200 μA aliquots. Since the density of HDL is <1.21 g/ml, HDL particles will be concentrated in the upper layers of the gradient. The top five 1 ml fractions with d<1.21 g/ml (HDL-rich) were recovered and pooled. The volume of the pooled HDL was concentrated to one half the original serum volume and the KBr removed using Centricon centrifugal filter units (10,000 MWCO). During the concentration, the buffer was exchanged into PBS, pH 7.4 by two centrifugation steps at 1000 rpm for 30 minutes at 4° C.

Protocol 2: Gradient Density Isolation of HDL from Polyethylene Glycol (PEG) Precipitated Serum Using a Single 21 Hour Ultracentrifugation

Removing VLDL and LDL from Serum by Precipitation with PEG.

A 1.5 ml aliquot of the commercially supplied human serum pool (Bioreclamation, LLC) was mixed with 0.6 ml of a 20% PEG solution in 200 mM glycine (pH 7.46) into a 5 ml cryogen tube. The resulting 2.1 ml was split equally into two 1.5 ml Eppendorf tubes and allowed to sit undisturbed for 30 minutes at room temperature.

At the end of the 30 minutes, the 2 tubes were centrifuged at 10,000 rpm for 30 minutes at 4° C. to pellet the VLDL and LDL at the bottom of the tube; leaving a supernatant containing HDL and other serum proteins above. At the conclusion of the spin a total of approximately 1.9 ml of the HDL-containing supernatant was collected and pooled. An aliquot of 125 μl of the supernatant was removed and stored at 4° C. for future analysis. The remaining 1.775 ml volume of supernatant was brought up to a volume of 4.5 ml with d=1.063 g/ml salt solution to match the volume of HDL fraction collected following the first ultracentrifugation of Protocol #1.

Adjusting the Density of the 4.5 ml PEG Supernatant to d=1.21 g/ml with KBr.

The density of the 4.5 ml PEG supernatant was determined by weighing 3 separate 0.2 ml aliquots and using the average of the 3 weights as the density. The density of the gradient using this method was 1.038 g/ml. In preparation for gradient separation ultracentrifugation the density of 4.5 ml of PEG supernatant was adjusted to a density 1.21 g/ml with potassium bromide (KBr). The amount of KBr needed to achieve this density was calculated using the following formula:

$$Grams of KBr = \frac{Initial Volume ( Final Density - Starting Density )}{1 - ( 0.318 ( Final Density ) )}$$

The density of the 4.5 ml of PEG supernatant was increased from the starting density of 1.038 to the final density of 1.21 by solubilizing 1.25 mg of KBr the 4.5 ml.

Purification of HDL by Ultracentrifugation in d=1.21 g/ml Salt Gradient.

The 4.5 ml of d=1.21 g/ml PEG supernatant was transferred to the bottom of a 10 ml polycarbonate ultracentrifuge tube and was overlayed with 5.5 ml of d=1.21 g/ml salt gradient solution. The tube was placed in a type Ti70 rotor and centrifuged at 39,100 rpm for 21 hours at 4° C. in a Beckman Model L80 Ultracentrifuge. At the conclusion of the centrifugation, the 10 ml was progressively removed in 1 ml increments from top to bottom and the density of each gradient fraction was determined by weighing 200 μL aliquots. Since the density of HDL is <1.21 g/ml, HDL particles will be concentrated in the upper layers of the gradient. The top five 1 ml fractions with d<1.21 g/ml (HDL-rich) were recovered and pooled. The HDL in the pool was concentrated to one half the serum starting volume and the KBr removed using Centricon centrifugal filter units (10,000 MWCO). During the concentration, the buffer was exchanged into PBS, pH 7.4 by two centrifugation steps at 1000 rpm for 30 minutes at 4° C.

### Determination Total Cholesterol, Total Protein and Lipid Profile

Total protein concentration was determined using the Markwell modification of the Lowry method. Total Cholesterol was determined using Wako cholesterol kit in 96-well plate format. Separation and quantification of lipoprotein complexes was performed by agarose gel electrophoresis using SPIFE 2000 (Helena labs) instrument and QuickScan (Helena Labs) software.

### Results

Tables 1 and 2 summarize the total protein and lipoprotein contents of the starting human serum and material obtained from each isolation protocol.

Total Protein and Lipoprotein Content of Starting Pool of Human Serum

The starting human serum pool sample contained of 7200 mg/dL protein and total cholesterol of 262 mg/dL. Separation of the lipoproteins by agarose electrophoresis (SPIFE) indicated the following lipoprotein composition: HDL-C 48 mg/dL (18%); Lp(a) 2.1 mg/dL (0.8%); VLDL-C 33.6 mg/dL (13%) and LDL-C 178 mg/dL (68%). Therefore in 1.5 ml of starting serum volume there is 0.72 mg of HDL-C and 108 mg protein.

Protocol #1 Recovery and Purity of HDL Isolated from Human Serum by 2 Spin Density Gradient Separation and Spin Filter Capture.

Material obtained from Protocol #1 after concentration and buffer exchange had a total protein concentration of 570 mg/dL and total cholesterol of 37 mg/dL. The lipoprotein composition determined by agarose gel electrophoresis was: HDL-C 27 mg/dL (80%); Lp(a) 0.4 mg/dL (0.4%); VLDL-C 0.2 mg/dL (0.6%) and LDL-C 6.8 mg/dL (18%). The yield of HDL-C was 0.20 mg and the yield of protein was 4.3 mg. After accounting for volume changes during each step of Protocol #1, the recovery of HDL-C from the starting human serum pool was 35%. However as indicated from the electrophoresis of the lipoproteins, the purity of HDL-C in this preparation using Protocol #1 was ˜80%.

Protocol #2 Recovery and Purity of HDL Isolated from Human Serum by PEG Precipitation and Single Spin Density Gradient Separation and Spin Filter Capture.

The supernatant collected following PEG precipitation in step 1 had a total protein of 5400 mg/dL and total cholesterol of 33 mg/dL. Separation of the lipoproteins by agarose gel electrophoresis (SPIFE) demonstrated the following lipoprotein composition: HDL-C 33 mg/dL (99.6%) with negligible (<0.1%) presence of Lp(a), VLDL and LDL. After accounting for volume changes during the PEG precipitation step, the recovery of HDL-C from the starting human serum pool was 97%. The relatively high level of protein in the PEG supernatant compared to the starting serum is due to the presence of lipid-free serum proteins that remain in the supernatant.

Material obtained from Protocol #2 after KBr centrifugation, concentration and buffer exchange had a protein concentration of 940 mg/dL and total cholesterol of 27 mg/dL. The lipoprotein composition determined by agarose gel electrophoresis was: 27 mg/dL HDL-C (99.8%). There was negligible (<0.1%) presence of Lp(a), vLDL and LDL. After accounting for volume changes during each step of protocol #2, the recovery of HDL-C from the starting human serum pool was 37%. Most importantly, electrophoresis of the lipoproteins isolated by protocol #2 indicated that this material was >99% pure HDL. The greater reduction in protein concentration in the HDL preparation from protocol #2 is due to the removal of the other lipid-free serum proteins. Therefore, the method of the present invention (Protocol #2) yields HDL-C of much greater purity than the standard sequential ultracentrifugation method (Protocol #1), 99.8% vs 80%, respectively.

### Discussion

While both methods for HDL isolation yielded identical concentrations of HDL-C (27 mg/dL) and similar recovery (35-37%) of HDL-C from human serum, the material obtained from the standard sequential density gradient ultracentrifugation protocol also contained 6.8 mg/dL LDL resulting in a HDL-C purity of 80%. In contrast, material obtained from the single density gradient ultracentrifugation procedure using PEG-precipitated serum was essentially pure HDL (>99%). These data indicate that PEG precipitation of human serum eliminated the need for the 18 hour ultracentrifugation required in the first step of the standard HDL isolation protocol. In addition to eliminating more than 17 hours from the procedure, our data indicated that the improved method using PEG precipitation actually increased the purity of the HDL recovered. Therefore, our method produces benefits in both processing time and quality of recovered HDL when compared to standard HDL isolation protocols.

TABLE 1
Lipoprotein profile of starting human serum pool and material
obtained from each HDL isolation protocol.

| | Starting | PEG | | |
|---|---|---|---|---|
| | Serum | Supernatant | Protocol 1 | Protocol 2 |
| Total | 262 | 33 | 37 | 27 |
| Cholesterol | | | | |
| (mg/dl) | | | | |

| | | mg/ | | | | | | |
|---|---|---|---|---|---|---|---|---|
| | % | dL | % | mg/dL | % | mg/dL | % | mg/dL |
| HDLc | 18 | 48 | >99 | 33 | 80 | 27 | >99 | 27 |
| Lipo- | 0.8 | 2.1 | 0.2 | — | 1.1 | 0.4 | 0.1 | — |
| protein | | | | | | | | |
| in (a) | | | | | | | | |
| VLDLc | 13 | 34 | 0.1 | — | 0.6 | 0.2 | 0 | — |
| LDLc | 68 | 178 | 0.1 | — | 18 | 6.8 | 0.1 | — |

TABLE 2
Biochemical profile and recovery and purity
of HDL isolated by each protocol.

| | Total | Total | | | |
|---|---|---|---|---|---|
| | Cholesterol | Protein | HDL-C | HDL-C % | HDL-C |
| Sample | mg/dL | mg/dL | mg/dL | Recovery | % Purity |
| Human | 263 | 7200 | 48 | — | — |
| Serum | | | | | |
| Protocol #1 | 37 | 570 | 27 | 35 | 80 |
| PEG | 33 | 5400 | 33 | 97 | >99 |
| Supernatant | | | | | |
| Protocol #2 | 27 | 940 | 27 | 37 | >99 |

In summary, the present invention provides a methodology for the isolation of HDL or HDL-like particles from human serum that requires substantially less time and resources and provides a higher purity HDL or HDL-like particles than was achieved from a standard HDL isolation protocol.

We claim:

1. A method of isolating high density lipoproteins or high density lipoprotein-like particles from a bodily fluid or homogenized tissue comprising:
a. adding one or more reagents to the bodily fluid or homogenized tissue;
b. ultracentrifugation of the bodily fluid or homogenized tissue; and
c. recovering the high density lipoproteins or high density lipoprotein-like particles.

2. The method of claim 1, wherein the one or more reagents bind and precipitate apoprotein B-containing proteins.

3. The method of claim 2, wherein the apoprotein B-containing proteins are low density lipoproteins and very low density lipoproteins.

4. The method of claim 1, wherein the ultracentrifugation of the bodily fluid or homogenized tissue is a single ultracentrifugation of the bodily fluid or homogenized tissue.

5. The method of claim 1, wherein the bodily fluid or homogenized tissue is human bodily fluid or human homogenized tissue.

6. The method of claim 5, wherein the human bodily fluid is plasma, serum, lymph, cerebral spinal fluid or peritoneal lavage fluid.

7. The method of claim 1, wherein the bodily fluid is plasma, serum, lymph, cerebral spinal fluid or peritoneal lavage fluid.

8. The method of claim 1, wherein the one or more reagents is polyethylene glycol.

9. The method of claim 1, wherein the one or more reagents is an anti-apoprotein B antibody.

10. The method of claim 1, wherein the one or more reagents is tetracycline.

11. The method of claim 1, wherein the one or more reagents is not sulfated polysaccharides.

12. A method of isolating high density lipoproteins or high density lipoprotein-like particles from a bodily fluid or homogenized tissue comprising:
a. adding one or more reagents to the plasma or serum to bind and precipitate apoprotein B-containing proteins;
b. centrifugation of the plasma or serum to pellet the apoprotein B-containing proteins;
c. adjusting the density of the apoprotein B-containing proteins-free plasma or serum;
d. ultracentrifugation of the apoprotein B-containing proteins-free plasma or serum; and
e. recovering the high density lipoproteins or high density lipoprotein-like particles.

13. The method of claim 12, wherein the one or more reagents is polyethylene glycol.

14. The method of claim 12, wherein the one or more reagents is an anti-apoprotein B antibody.

15. The method of claim 12, wherein the one or more reagents is tetracycline.

16. The method of claim 12, wherein the one or more reagents is not sulfated polysaccharides.

17. A method of isolating high density lipoproteins or high density lipoprotein-like particles from a bodily fluid or homogenized tissue comprising:
a. ultracentrifugation of the bodily fluid or homogenized tissue to remove non-lipoprotein associated components of the bodily fluid or homogenized tissue;
b. adding one or more reagents to the plasma or serum to bind and precipitate apoprotein B-containing proteins; and
c. recovering the high density lipoproteins or high density lipoprotein-like particles.

18. The method of claim 17, wherein the one or more reagents is polyethylene glycol.
